(19) United States
(12) Patent Application Publication
(10) Pub. No.: US 2018/0200190 A1
(43) Pub. Date: Jul. 19, 2018

(54) MULTIPLE PILL ABUSE-RESISTANT IMMEDIATE RELEASE SOLID DOSAGE FORM OF HYDROCODONE

(71) Applicant: SUN PHARMA ADVANCED RESEARCH COMPANY LTD., Andheri (East), Mumbai (IN)

(72) Inventors: Nitin B. DHARMADHIKARI, Andheri (IN); Yashoraj R. ZALA, Andheri (IN); Bramhanand HANAMANNAVAR, Andheri (IN)

(73) Assignee: SUN PHARMA ADVANCED RESEARCH COMPANY LTD., Andheri (East), Mumbai (IN)

(21) Appl. No.: 15/742,739

(22) PCT Filed: Jul. 8, 2016
(86) PCT No.: PCT/IN2016/050227

§ 371 Date: Jan. 8, 2018

(30) Foreign Application Priority Data
Jul. 10, 2015 (IN) 2628/MUM/2015

(51) Int. Cl. A61K 9/20; A61K 31/485; A61K 47/32

(52) U.S. Cl. CPC A61K 9/2013; A61K 9/2027; A61K 9/2095; A61K 31/167; A61K 47/32; A61K 2300/00; A61K 31/485

(57) ABSTRACT

An immediate-release, abuse-resistant solid dosage form comprising melt extrudates of a mixture of:
hydrocodone or its pharmaceutically acceptable salt,
release inhibiting agent comprising one or more reverse enteric polymer and one or more alkalizer and
one or more stabilizers.

### FIELD OF INVENTION

The present invention relates to abuse-resistant, immediate-release solid dosage of an opioid derivative such as hydrocodone and/or its combination with one or other active ingredient. The present invention also relates to a method of providing resistance to abuse achieved by various modes, such as extraction and overdosing.

### BACKGROUND OF INVENTION

Hydrocodone/Acetaminophen, with over 135 million dispensed prescriptions in 2014 in the United States, is the largest prescribed to treat moderate to severe pain. It also is the single most abused prescription, predominately by swallowing multiple tablets, apart from other means of abuse such as tampering, extraction and snorting. It is known that chronic abuser generally may consume multiple doses of an immediate or rapid release dosage form. Therefore, dosage forms that release a drug susceptible to abuse rapidly are more prone to abuse by administration of multiple pills. To date, there is no approved, available solid dosage form of a drug susceptible to abuse, that allows resistance to multiple pill administration and provides deterrence to its abuse.

There are a plethora of references describing solid dosage form of hydrocodone that shows resistance to abuse by tampering, extraction or snorting or the like. One such reference, namely, United States Patent Application Number US20140155388 discloses an abuse resistant solid dosage form of opioid derivatives by incorporating an acid soluble ingredient and a buffering ingredient. Another reference, United States Patent Application Number US20150017240 describes an abuse resistant solid dosage form by coating the drug with acid soluble ingredient. However, to date, there are no abuse proof dosage forms of hydrocodone that present resistance to multiple pill abuse apart from the other means of abuse, such as tampering by crushing, extraction and snorting. During the development of the multiple pill abuse-resistant, immediate-release solid dosage form as described in co-pending United States patent application numbers U.S. Ser. No. 14/667,834 and U.S. Ser. No. 14/667,826 which have been incorporated herein by reference, the inventors have found that the use of a release inhibiting agent such as an alkalizer was particularly advantagenous, particularly when included partly intragranularly in admixture with the drug that is susceptible to abuse, and partly in the extragranular phase. However, while developing an abuse-resistant solid dosage form of hydrocodone, the inventors faced a problem of chemical instability, particularly, when the preparation of the intragranular phase involved steps of application of heat such as hot melt extrusion. This problem was never recognized previously in the prior art. This problem was solved by use of a stabilizer such as a fusible material in the intragranular phase in admixture with hydrocodone, along with the release inhibiting agent which is a mixture of reverse enteric polymer and one or more alkalizer. The solid dosage form so prepared, was very robust in terms of chemical stability.

### SUMMARY OF THE INVENTION

The present invention provides an immediate-release, abuse-resistant solid dosage form comprising melt extrudates comprising a mixture of
a) hydrocodone or its pharmaceutically acceptable salt.
b) release inhibiting agent comprising one or more reverse enteric polymer and one or more alkalizer and
c) one or more stabilizers.

Particularly, the melt extrudates are chemically stable and when incorporated into a solid dosage form, the dosage form is stable.

The present invention also provides a process of forming melt extrudates by mixing the hydrocodone or its pharmaceutically acceptable salt and reverse enteric polymer followed by heating the mixture to a temperature of from 60° C. to 220° C. to prepare an immediate release abuse resistant solid dosage form, a method of reducing degradation of hydrocodone resulting from the process, the method of reducing degradation of hydrocodone comprising performing the melt extrusion by subjecting the mixture of:
a) hydrocodone or its pharmaceutically acceptable salt,
b) release inhibiting agent comprising one or more reverse enteric polymer and one or more alkalizer and
c) one or more stabilizers;
   followed by heating the mixture to a temperature of from 60° to 220° C.

### DETAILED DESCRIPTION OF THE INVENTION

The term ‘stable’ as used herein means either the melt extrudates or solid dosage form when stored for a duration of three months at 40° C. and relative humidity of 75% or for a duration of six months at 30° C. and relative humidity of 65% or for a duration of six months at 25° C. and relative humidity of 60%; the single highest individual unspecified impurity is less than 0.2% by weight of the hydrocodone and the individual known impurity is less than 0.33% by weight of the hydrocodone, when determined by high performance liquid chromatography.

The term ‘stabilizer’ as used herein means that a compound or pharmaceutically acceptable material that prevents the degradation of hydrocodone or its pharmaceutically acceptable salt, when present in admixture with hydrocodone, while hydrocodone is being subjected to melt extrusion. It was found that when the melt extrudates or the solid dosage form were prepared without such stabilizers, hydrocodone degraded to unacceptable levels of either known or unspecified chemical impurities. Without wishing to be bound by any theory, it is understood that hydrocodone may be degrading due the presence of reverse enteric polymer alone, which needs to be incorporated in the melt extrudates to provide abuse resistance to the solid dosage form. It was found that when a stabilizer is incorporated along with the hydrocodone and release inhibiting agent, the solid dosage form remains substantially resistant to degradation. This degradation may be attributed to the interaction of the hydrocodone with the degradative compounds itself, which may be the reverse enteric polymer or the alkalizer or the combination thereof, or due to the conditions under which the melt extrusion is carried out.

In one embodiment, the hydrocodone in the melt extrudates is substantially resistant to formation of impurities resulting from a chemical change in the hydrocodone brought about during the production and/or storage of the solid dosage form having the melt extrudates. Statutory requirements, including, but not limited to, ICH (International conference on harmonisation) guidelines Q3A and Q3B, identify maximum allowable amounts of degradants above which the degradants must be reported and subjected to the quantification process after the solid form has been stored for 6 months at 40° C. and 75% relative humidity.

The determination and quantification may be done by any known methods. It may be determined by high performance liquid chromatography with a gradient programmed flow rate of 1 mL/minute using mobile phase A which is mixture of potassium phosphate monobasic and 1-octanesulfonic acid sodium salt, monohydrate in purified water and mobile phase B which is 100% acetonitrile using a ODS (octadecyl Silane) column. The known impurities of hydrocodone that are detected and quantified are hydrocodone N-Oxide and the aldol dimer impurity. These individual known impurities of hydrocodone are less than 0.33% by weight of the hydrocodone, when determined by high performance liquid chromatography. The individual highest unspecified impurity is the impurity for which the structure is not known but requires detection and quantification below certain limits as defined by statutory requirements, including, but not limited to, ICH guidelines Q3A and Q3B, identify maximum allowable amounts of degradants above which the degradants must be reported and subjected to the quantification process after the solid form is being stored for 6 months at 40° C. and 75% relative humidity. The melt extrudates or the solid dosage form is said to be stable when any unknown or unspecified highest impurity of hydrocodone is detected and is not more than 0.2% by weight of hydrocodone, when determined by high performance liquid chromatography. Accordingly, the present invention provides an immediate-release, abuse-resistant solid dosage form wherein the immediate-release, abuse-resistant solid dosage form is said to be stable in that the single individual unspecified impurity in the solid dosage form does not increase to more than 0.2% by weight of hydrocodone bitartarate and the individual known impurity of hydrocodone is less than 0.33% of weight of hydrocodone bitartarate, when the dosage form stored in containers for a duration of three months at 40° C. and relative humidity of 75% or for a duration of six months at 30° C. and relative humidity of 65% or for a duration of six months at 25° C. and relative humidity of 60%; wherein the level of impurities is determined by high performance liquid chromatography.

In one embodiment, the salt of hydrocodone is a bitartrate salt and it is present in the range from about 1 mg to 100 mg per unit dosage form, such as 5, 10, 15, 20, 25, 30, 35, 40, 45, 50, 55, 60, 65, 70, 75, 80, 85, 90 or 95 mg, particularly 5 mg to 20 mg. More preferably the amount of hydrocodone in the immediate release multiple pill abuse resistant solid dosage form is present in the range of 1 to 50 mg per unit dosage form, most preferably in the range of 1 to 20 mg per unit dosage form.

In one embodiment the hydrocodone bitartrate is present in an amount of 2.5 mg. In another embodiment the hydrocodone bitartrate is present in an amount of 5 mg. In yet another embodiment, hydrocodone bitartrate is present in an amount of 7.5 mg. In one exemplary embodiment, hydrocodone is present in an amount of 10 mg per unit dosage form.

The hydrocodone present as its salt, may be present in a range of 0.1 to 50% such as 0.5%, 1%, 1.5%, 2% 2.5%, 3% 3.5% 4% 4.5% 5% 5.5% 10% 10.5%, 15%, 15.5% 20% 20.5%, 25% 25.5%, 30% 30.5%, 35%, 35.5%, 40% 40.5%, 41%, 42%, 45%, 50% by weight of the melt extrudates, more preferably in the range of 1 to 20% by weight of the melt extrudates and most preferably, in the range of 5% to 10% by weight of the melt extrudates.

The stabilizer is a fusible material according to the present invention. The term ‘fusible’ is intended to mean that the polymer can melt on heating without undergoing degradation, which is a vinyl containing polymer. Suitable examples of the vinyl containing polymer, include, but are not limited to, polyvinyl acetate, polyvinyl alcohol, polyvinylcaprolactam-polyvinyl acetate-polyethylene glycol graft co-polymer and vinylpyrrolidone-vinyl acetate copolymers, copolymers of N-vinyl pyrrolidone and vinyl acetate or vinyl propionate, polyvinyl pyrrolidone, polyvinyl alcohol-polyethylene glycol-graft copolymers (available as Kollicoat® IR from BASF AG, Ludwigshafen, Germany); vinyl acetate polymers such as copolymers of vinyl acetate and crotonic acid, partially hydrolyzed polyvinyl acetate (also referred to as partially saponified “polyvinyl alcohol”).

In certain embodiments, ancillary stabilizers may be included along with the stabilizers that are fusible that is a polymer that can melt on heating without undergoing degradation. Such ancillary stabilizers may be antioxidant in nature. Examples of the suitable ancillary stabilizers which is an antioxidant may be tartaric acid, gallic acid or its derivatives, butylated hydroxyl toluene or butylated hydroxyl anisole. It was found that presence of compounds like polyethylene derivatives, such as polyethylene glycol, polyethylene oxides, in fact destabilized the hydrocodone, inspite of presence of stabilizers such as polyvinyl acetate, polyvinyl alcohol, polyvinylcaprolactam-polyvinyl acetate-polyethylene glycol graft co-polymer and vinylpyrrolidone-vinyl acetate copolymers, copolymers of N-vinyl pyrrolidone and vinyl acetate or vinyl propionate, polyvinyl pyrrolidone, polyvinyl alcohol-polyethylene glycol-graft copolymers (available as Kollicoat® IR from BASF AG, Ludwigshafen. Germany); vinyl acetate polymers such as copolymers of vinyl acetate and crotonic acid, partially hydrolyzed polyvinyl acetate (also referred to as partially saponified “polyvinyl alcohol”) with and without presence of antioxidants such as butylated hydroxyl anisole or organic acids like citric acid, formic acid, tartaric acid and the like and mixtures thereof. In one preferred embodiment, melt extrudates of the immediate-release, abuse-resistant solid dosage form contains stabiliser in amount ranging from 1 to 20% such as 1%, 2%, 3%, 4%, 5%, 6%, 7%, 8%, 9%, 10%, 11%, 12% 13%, 14%, 15%, 16%, 17%, 18%, 19%, 20% by weight of the melt extrudates. In one specific embodiment, the stabilizer is polyvinyl alcohol. In another embodiment, the stabilizer is a mixture of partially hydrolysed polyvinyl alcohol, butylated hydroxyl anisole and tartaric acid. In another preferred embodiment the immediate-release, abuse-resistant solid dosage form has partially hydrolysed polyvinyl alcohol present in an amount ranging from 1 to 20% by weight of the melt extrudate, butylated hydroxyl anisole present in an amount ranging from 0.1 to 0.5% such as 0.1%, 0.2%, 0.3%, 0.4%, 0.5% by weight of the melt extrudates and organic acid present in amount ranging from 1 to 10% such as 1%, 2%, 3%, 4%, 5%, 6%, 7%, 8%, 9%, 10% by weight of the melt extrudates as the stabiliser and hydrocodone bitartrate present in an amount ranging from 1 to 20% such as 1%, 2%, 3%, 4%, 5%, 6%, 7%, 8%, 9%, 10%, 11%, 12%, 13%, 14%, 15%, 16%, 17%, 18%, 19%, 20% by weight of the melt extrudates.

In one specific embodiment, the weight ratio of hydrocodone bitartarate to reverse enteric polymer is 1:1 to 1:20, such as 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20. In one specific embodiment, when the stabilizer is polyvinyl alcohol, the weight ratio of hydrocodone bitartarate to polyvinyl alcohol is 1:0.1 to 1:10.

The melt extrudates in the solid dosage form of the present invention comprises release inhibiting agent, along with hydrocodone or its pharmaceutical salt and a stabilizer. The ‘release inhibiting agent’ as used herein refers to a substance or a combination thereof, that functions to inhibit the release of the drug susceptible to abuse in gastric fluids only when more than the prescribed number of units of the dosage form are orally administered, or has no effect. In this way, the dosage form of the present invention is useful to deter the abuse of hydrocodone or its pharmaceutically acceptable salt by drug addicts.

In certain embodiments, where the prescribed number of units of the immediate release multiple pill abuse resistant solid dosage form of the present invention is two, then the composition of the release inhibiting agent used is such that the two prescribed number of units provide the release of the hydrocodone or its pharmaceutically acceptable salt which is equivalent to the release obtained from the conventional immediate release dosage form. But, when three or more number of units is tested, the release is inhibited as compared to the equivalent number of units of the conventional immediate release solid dosage form. It is observed that as the number of units of the immediate release multiple pill abuse resistant solid dosage form of the present invention increases, release rate decreases. This will provide deterrence particularly, against misuse, intentional such as suicidal (overdose) or unintentional, or abuse by an abuser or addict.

The term. “reverse enteric polymer” as used herein refers to a polymer that is soluble in acidic solutions but is insoluble or alternatively swells or gels above a second higher pH value. Whether a polymer is insoluble above the second pH value is determined as follows: 500 mg of the reverse enteric polymer is dispersed in 100 ml of 0.05 N HCL and its pH adjusted to the second pH value by adding an alkali. Percentage transmission of the dispersion is measured at 260 nm. The reverse enteric polymer is defined as ‘insoluble’ at and above the second specific pH value, if the percentage transmission obtained at the second pH value is below 70%. The reverse enteric polymer used as the release inhibiting agent along with one or more alkalizer, is selected from polymers that are prepared by polymerizing a mixture of the hydrophobic and basic monomer or a mixture of the hydrophobic, hydrophilic and basic monomer wherein the basic monomer may be selected from the group consisting of dimethyl amino ethyl acrylate, diethyl amino ethyl ethacrylate, diethyl amino ethyl acrylate, piperidine ethyl methacrylate and 2-tert-butyl amino ethyl methacrylate. The “reverse enteric polymer” of the release inhibiting agent used in the immediate-release, abuse-resistant solid dosage form of the present invention is a polymer that is soluble in acidic solutions but is insoluble or alternatively swells or gels above a second higher pH value. In several of the embodiments, as herein described, the reverse enteric polymer functions as a release rate controlling polymer above a critical pH but has little rate controlling ability below the critical pH. Examples are found in polymers that have group capable of accepting the hydrogen ion from an acid below the critical pH and thus becoming soluble in acid environment and thus fall under the class of pH dependent polymers. An example of a preferred reverse enteric polymer i.e a pH dependent polymer used is a methyl methacrylate butyl methacrylate-dimethyl aminoethyl methacrylate copolymer which is a cationic copolymer synthesized from dimethyl aminoethyl methacrylate and neutral methacrylic acid esters, more particularly as is commercially available under the trade name Eudragit™ E which is soluble below an acidic pH such as pH 5 and swellable and permeable above about a higher pH such as above 5.0. The Eudragit™ E 100 product is granular, the Eudragit™ E 12.5 product is a 12.5% solution of E 100 in isopropanol and acetone, and the Eudragit EPO product is a fine powder made from E 100. Various grades of this polymer are commercially available from Evonik, Germany. The amount of Eudragit™ E in the present invention varies from 0.5% to about 30% by weight of the solid dosage form, preferably about 2% to about 30% by weight of the solid dosage form, more preferably about 5% to about 20% by weight of the solid dosage form. The ratio of weight of polymer to the weight of hydrocodone or its pharmaceutically acceptable salt varies from 0.5 to about 8.0 such as 0.5, 0.75, 1, 1.25, 1.5, 1.75, 2, 2.5, 2.75, 3, 3.5, 3.75, 4, 4.5, 5, 5.75, 6, 6.75, 7, 7.5, 8, preferably about 3.0 to about 8.0, more preferably about 6.8. The amount of this reverse enteric polymer may be expressed in terms of its weight ratio.

Suitable examples of the reverse enteric polymer of the release inhibiting agent that is soluble at an acidic but is insoluble at a second higher pH value, include, but are not limited to, methyl methacrylate and diethylaminoethyl methacrylate and the like. Any other reverse enteric polymer having such properties is encompassed within the scope of this embodiment of the present invention. In one specific preferred embodiment, the reverse enteric polymer that can be utilized in the present invention is a copolymer comprising amino and/or alkylamino and/or dialkyl amino groups such as copolymers comprising methyl methacrylate and diethylaminoethyl methacrylate such as commercially available as Kollicoat® Smartseal 30 D from BASF. The polymer has a molecular weight of about 200,000 and a glass transition temperature of 57 to 63° C. The weight ratio of hydrocodone or its pharmaceutically acceptable salt to the reverse enteric polymer varies in the range from 1:6 to 1:50 such as 1:7, 1:8, 1:9, 1:10, 1:11, 1:12, 1:13: 1;14, 1:15, 1:16, 1:17, 1:18, 1:19, 1:20, 1:21, 1:22, 1:23, 1:24, 1:25, 1:26, 1:27, 1:28, 1:29, 1:30, 1:32, 1:33, 1:34, 1:35, 1:36, 1:37, 1:38, 1:39, 1:40, 1:41, 1:42, 1:45 1:46, 1:47, 1:48, 1:47, 1:50, preferably in the range of 1:12 to 1:48. In the preferred embodiment, the weight ratio of hydrocodone or its pharmaceutically acceptable salt to the reverse enteric polymer is 1:12.

The term ‘alkalizer’ as used herein means any agent that suppresses the gastric acid environment. The alkalizer may work by physicochemical mechanisms that result in inhibition of in-vitro release as well as in-vivo release. For example, an alkalizer can increase the pH by neutralization of acid. The amount of alkalizer used in a single unit is selected so that it will not be sufficient to raise the stomach pH to above a critical pH for example 5 or neutral pH; but when more than the prescribed number of units are administered, it is sufficient to raise the pH of the stomach to above the critical pH. Usually the amount of alkalizer when more than the prescribed number of units should at least raise the pH of 500 ml of 0.01 N HCl to above the critical pH, preferably the amount should be greater and raise the pH of 1000 ml of 0.01N HCl to above the critical pH, and more preferably it may exceed that amount sufficiently to neutralize any immediate rebound secretion of acidic gastric fluids in response to the alkalizer. The amount of alkalizer in one single unit is however selected so that it does not raise the pH of 500 ml, preferably 1000 ml of 0.01N HCl to above the critical pH so that when a single unit is orally administered the polymer does not behave like a rate controlling polymer but when more than the prescribed number of units are administered, it behaves like a rate controlling polymer and inhibits release. The alkalizer of the release inhibiting agent that is present in melt extrudates or the extragranular phase of the immediate-release, abuse-resistant solid dosage form may be selected from a group consisting of, but are not limited to, calcium carbonate, disodium hydrogen phosphate, trisodium orthophosphate, sodium hydroxide, sodium carbonate, potassium hydroxide, sodium bicarbonate, dipotasium carbonate, tromethamine, aluminium trihydroxide, magnesium dihydroxide, aluminium oxide, magnesium oxide and mixtures thereof. The amount of alkalizer present in the melt extrudates ranges from 5% to 15% such as 5%, 5.5%, 6%, 7%, 8%, 8.5%, 9%, 9.5%, 10%, 10.5%, 11%, 11.5%, 12%, 13%, 13.5%, 14%, 15% by weight of the melt extrudates, preferably, about 10% by weight of the melt extrudates. The ratio of the alkalizer present in the extragranular phase to the alkalizer present in the intragranular phase is about 5 to 20 such as 5, 6 7, 8, 10, 12, 14, 16, 18, 20. The alkalizer used in the solid dosage form of the present invention is present in the range of 10 to 40% such as 11%, 12%, 13%, 14%, 15%, 18%, 20%, 25%, 30%, 35%, 40% by weight of the total dosage form. More preferably the alkalizer is present in the range of 14 to 35% by weight of the solid dosage form. In one embodiment, it is possible to include a water insoluble alkalizer in the melt extrudates and a mixture of water insoluble and water soluble alkalizers in the extragranular phase. In another embodiment, it is possible to include a water soluble alkalizer in the melt extrudates and a mixture of water insoluble and water soluble alkalizers in the extragranular phase or the water soluble alkalizer alone, in the extragranular phase. In another embodiment, it is possible to use a mixture of water insoluble and water soluble alkalizers in the intragranular phase, which is the melt extrudates.

According to the present invention, the melt extrudates having a mixture of hydrocodone or its pharmaceutically acceptable salt, release inhibiting agent which is a mixture of reverse enteric polymer and alkalizer and a stabilizer, form the intragranular phase of the solid dosage form. Such a solid dosage form additionally may include a second active ingredient either as a separate intragranular phase or may be present in the non-granular form, that is the active ingredient as such, as a physical admixture with other inert excipients.

In one specific embodiment, an immediate release, abuse resistant solid dosage form comprises melt extrudates which is mixture of:
a, hydrocodone or its pharmaceutically acceptable salt as sole active ingredient,
b, release inhibiting agent comprising one or more reverse enteric polymer and one or more alkalizer and
c, one or more stabilizers,
   and these melt extrudates are present in the form of first intragranular phase, wherein the melt extrudates are free of polyethylene glycol or polyethylene oxide or similar compounds. This first intragranular phase may be mixed with another intragranular phase comprising second active ingredient. The first and the second intragranular phase is further mixed with extragranular phase having an alkalizer. It is also possible to incorporate the second active ingredient in the extragranular phase in the non-granular phase along with the alkalizer. The solid dosage form may optionally, contain second active ingredient which may be any other non-opioid analgesic. Suitable examples, include, hut are not limited to, acetaminophen, aspirin, bromfenac, diclofenac, diflunisal, etodolac, fenoprofen, flurbiprofen, ibuprofen, indomethacin, ketoprofen, ketorolac, meclofenamate, mefenamic acid, meloxicam, nabumetone, naproxen, oxaprozin, phenylbutazone, piroxicam, sulindac, tolmetin, celecoxib.

In one preferred embodiment, the non-opioid analgesic is acetaminophen. In the embodiment with acetaminophen, the solid dosage form may contain acetaminophen ranging from about 50 mg to 500 mg such as 50 mg, 55 mg, 60 mg, 62, mg, 63 mg, 65 mg, 68 mg, 70 mg, 75 mg, 76, mg 78 mg, 80 mg, 85 mg, 89 mg, 90 mg, 95 mg, 100 mg, 105 mg, 115 mg, 125 mg, 130 rug, 140 mg, 150 mg, 160 mg, 175 mg, 180 mg, 185 mg, 190 mg, 195 mg, 200 mg, 205 mg, 206 mg, 210 mg 250 mg, 260 mg, 270 mg, 280 mg, 290 mg, 300 mg, 310 mg, 315 mg, 320 mg, 330 mg, 340 mg, 350 mg, 360 mg, 370 mg, 380 mg, 390 mg, 400 mg, 410 mg, 420 mg, 450 mg, 470 mg, 490 mg, 500 mg per unit of the solid dosage form. In one embodiment, the acetaminophen is present in an amount of 300 mg. In the preferred embodiment the acetaminophen is present in a concentration of 325 mg per unit of the immediate release multiple pill abuse resistant solid dosage form. In one embodiment, the acetaminophen present in a second intragranular phase, in the form of granules having a particle size such that a minimum of 80% granules have particle size greater than 45 microns, around 75 to 95% of the granules have a size greater than 75 microns, around 70% of the granules have a size greater than 150 microns and about 30% of the granules have a granule size greater than 250 microns.

In one specific embodiment, the melt extrudates comprising mixture of hydrocodone or its salt, release inhibiting agent having reverse enteric polymer and an alkalizer and polyvinyl alcohol as the sole stabilizer. These melt extrudates provided adequate stability in that the known and individual unspecified impurity values were within the acceptable limit when checked immediately after extrusion. However, when these melt extrudates were formulated into a solid dosage form wherein the extrudates form the intragranular phase with extragranular phase of alkalizer and another active ingredient, the solid dosage form provided unacceptable limits of the known impurities, such as hydrocodone N-oxide or aldol dimer impurity. When the melt extrudates included a combination of stabilizers such as a fusible material like polyvinyl alcohol, an antioxidant like butylated hydroxyl anisole and tartaric acid, and when the melt extrudates were formulated into a solid dosage form wherein the extrudates form the intragranular phase with extragranular phase of alkalizer and another active ingredient, surprisingly, both the hydrocodone N-oxide and aldol dimer impurity levels were adequately controlled. Such solid dosage form was found to be stable and was complying with the ICH guidelines. In such embodiment, the melt extrudates contain one or more alkalizer selected from magnesium oxide, tromethamine, meglumine, calcium carbonate, sodium carbonate, sodium bicarbonate or mixtures thereof. Hydrocodone or its pharmaceutically acceptable salt is present in the amounts ranging from 5 mg to 20 mg per unit dosage form, wherein polyvinyl alcohol is present in admixture with hydrocodone or its pharmaceutically acceptable salt and methyl methacrylate and diethylaminoethyl methacrylate copolymer, a reverse enteric polymer. Along with polyvinyl alcohol, the melt extrudates further contain a mixture of at least one organic acid and butylated hydroxyl anisole. The organic acid may be selected from tartaric acid, citric acid, ascorbic acid or fumaric acid. The second composition with which the hot melt extrudates is admixed, may be a known composition of acetaminophen, naproxen, diclofenac or ibuprofen or its pharmaceutically acceptable salt.

It was found that increased degradation of hydrocodone when exposed to high temperatures in a hot melt extrusion with a reverse enteric problem which was solved by inclusion of stabilisers such as polyvinyl alcohol. The present invention provides in a process, of forming melt extrudates by mixing the hydrocodone or its pharmaceutically acceptable salt and reverse enteric polymer followed by heating the mixture to a temperature of from 60° C. to 220° C. to prepare an immediate release abuse resistant solid dosage form, a method of reducing degradation of hydrocodone resulting from the process, the method of reducing degradation of hydrocodone comprising performing the melt extrusion by subjecting the mixture of:
a) hydrocodone or its pharmaceutically acceptable salt,
b) release inhibiting agent comprising one or more reverse enteric polymer and one or more alkalizer and
c) one or more stabilizers;
   followed by heating the mixture to a temperature of from 60° C. to 220° C.

Alternatively, the present invention provides in a process, of forming melt extrudates by mixing the hydrocodone or its pharmaceutically acceptable salt and reverse enteric polymer followed by heating the mixture to a temperature of from 60° C. to 220° C. to prepare an immediate release abuse resistant solid dosage form, a method of reducing degradation of hydrocodone resulting from the process, the method of reducing degradation of hydrocodone comprising performing the melt extrusion by subjecting the mixture of:
a) hydrocodone or its pharmaceutically acceptable salt,
b) release inhibiting agent comprising one or more reverse enteric polymer and one or more alkalizer and
c) one or more stabilizers;
   followed by heating the mixture to a temperature of from 60° C. to 220° C.

Alternatively, the present invention provides in a process, of forming melt extrudates by mixing the hydrocodone or its pharmaceutically acceptable salt and reverse enteric polymer followed by heating the mixture to a temperature of from 60° C. to 220° C. to prepare an immediate release abuse resistant solid dosage form, a method of reducing degradation of hydrocodone resulting from the process, the method of reducing degradation of hydrocodone comprising performing the melt extrusion by subjecting the mixture of:
a) hydrocodone or its pharmaceutically acceptable salt,
b) release inhibiting agent comprising one or more reverse enteric polymer and one or more alkalizer and
c) one or more stabilizers;
   followed by heating the mixture to a temperature of from 60° C. to 220° C. wherein the melt extrudates are free of polyethylene glycol or polyethylene oxide.

In specific embodiment, the mixture comprises hydrocodone salt and wherein the hydrocodone salt is a bitartarate and the step of heating is performed at a temperature of from 90° C. to 150° C. Particularly, the step of heating is performed while the mixture is in an extruder. The stabilizer comprises polyvinyl alcohol or mixture of partially hydrolysed polyvinyl alcohol, butylated hydroxy anisole and organic acid. It was found that the presence of certain excipients like polyethylene glycol, polyethylene oxide, may cause destabilization in that upon storage, the highest individual unspecified impurities raised beyond acceptable limits. This is because the melt extrudates containing polyethylene glycol along with polyvinyl alcohol, butylated hydroxyl anisole and tartaric acid, showed more than 0.2% of highest individual unspecified impurities. Thus, in preferred embodiments the stabilizers used in the melt extrudates are devoid of polyethylene derivatives such as polyethylene glycol or polyethylene oxide or mixtures thereof.

Particularly, in one specific embodiment, the stabilizer is partially hydrolysed polyvinyl alcohol and is present in amount ranging from 1 to 20% by weight of the immediate release abuse resistant solid dosage form, stabilizer is a mixture of partially hydrolysed polyvinyl alcohol, butylated hydroxy anisole and organic acid. The organic acid is selected from tartaric acid, citric acid, ascorbic acid or fumaric acid. In one specific embodiment, butylated hydroxyl anisole is present in amount ranging from 0.1 to 0.5% by weight of the immediate release abuse resistant solid dosage form and organic acid is present in amount ranging from 1 to 10% by weight of the immediate release abuse resistant solid dosage form, and wherein hydrocodone bitartrate is present in amount ranging from 1 to 20% by weight of the immediate release abuse resistant solid dosage form. In yet another embodiment, the reverse enteric polymer is a methyl methacrylate and diethylaminoethyl methacrylate copolymer.

In one embodiment, the intragranular phase of the solid dosage form is prepared by the process of hot melt extrusion. The method comprises the step of preparing the extrudates of hydrocodone or its pharmaceutically acceptable salt and further producing a dry mix blend of extrudates of hydrocodone or its pharmaceutically acceptable salt with a non-opioid analgesic which is further compressed into tablets. The extrudates of hydrocodone or its pharmaceutically acceptable salt are prepared by sifting and blending hydrocodone or its pharmaceutically acceptable salt with a reverse enteric polymer, and optionally an alkalizer and mixing them in a suitable blender. The blended ingredients may be then processed using hot melt extrusion at a suitable temperature at which the hydrocodone or its pharmaceutically acceptable salt, alkalizer, reverse enteric polymer and the stabilizer melts and fuses without the drug's degradation.

For hot melt extrusion process suitable extruders such as single screw extruders, intermeshing screw extruders, multi screw extruders, twin screw extruders and other screw elements for mixing and dispersing the melt as known in the art can be used. It will be appreciated that the working temperature used will be determined by the kind of extruder used. It is important to select a temperature that does not cause degradation or decomposition of the hydrocodone or its pharmaceutically acceptable salt, during processing. The operating temperature is generally in the broad range of about 60° C. to about 220° C. The hot melt extrudes of hydrocodone or its pharmaceutically acceptable salt thus formed are then dry mixed with the non-opioid granules, binder, glidant, disintegrant alkalizer, lubricant; are passed through suitable sieve and blended together and compressed using tablet press. In one embodiment the extrudates prepared by hot melt extrusion have a particle size distribution such that 34.82% of the extrudates have a particle size greater than 250 microns, 57.16% of the extrudates have a particle size distribution of greater than 180 microns, 66.8% of extrudates have a particle size greater than 150 microns, 74.16% of extrudates have a particle size distribution greater than 125 microns, 87.25% of extrudates have a particle size greater than 125 microns and all the extrudates have a particle size greater than 75 microns.

The solid dosage form of the present invention may be fabricated into a suitable form such as sachets, capsules or tablet by methods known in the art and using conventional excipients known in the art such as diluents or fillers, binders, disintegrants, stabilisers glidants, lubricants, surfactants, solubilizing agents, preservatives, colouring agents and others as may be necessitated by the drug to be incorporated in the dosage form.

The term ‘conventional immediate release dosage form’ as used herein means that the dosage form is a conventional dosage form that releases the drug rapidly but is not capable of providing resistance to abuse by known means such as ingestion of multiple units of the dosage form.

The present invention presents a method of providing resistance to abuse by at least one of the modes such as
a. intentional abuse of overdosing or multiple unit administration by an addict or by a subject,
b. intentional abuse of extraction from multiple unit administration by an addict or by a subject.
c. unintentional or accidental overdosing,
d. concomitant alcohol consumption and resultant drug-alcohol interaction
e. intentional abuse by nasal, parenteral, rectal or oral route
f. separating two phases by physical means with an intention to abuse
   wherein the immediate release multiple pill abuse resistant solid dosage form comprises
hydrocodone or its pharmaceutically acceptable salt,
release inhibiting agent comprising one or more reverse enteric polymer and one or more alkalizer and
one or more stabilizer.

The abuse resistant properties of the immediate-release abuse-resistant solid dosage form of the invention is adjudged using various tests such as extraction in various solvents, extraction of hydrocodone free base, cold water extraction. Besides other tests such as syringeability, Injectability and extraction in simulated nasal fluid were conducted to evaluate the resistance to abuse by a potential abuser employing various means to extract the drug. The reference used for the purpose of comparison was the hydrocodone bitartarate acetaminophen tablets commercially available as Norco™. The solid dosage form of the present invention in the form of tablets and reference NORCO™ were subjected to extraction studies to evaluate ease of extracting hydrocodone bitartrate (HCB) from the intact and manipulated formulation using commonly available household solvents namely water at room temperature, water at 8° C., water at 85° C., 0.9% saline at 85° C. For the test a suitable number of tablets were weighed and crushed in a coffee grinder. The entire contents were transferred into a suitable flask and extracted with different solvents and the content of hydrocodone analysed using assay methodology.

For the free base extraction test a suitable number of tablets of the solid dosage form prepared as per the present invention and the reference Norco™ were weighed and crushed in a coffee grinder and powder transferred to a suitable beaker. Warm water was added to the powdered sample and was kept in freezer for suitable time. The solution was slowly filtered and filtrate collected and pH adjusted to 8-9 with suitable solution. Hydrocodone precipitated out in the solution as free base. The dispersion was extracted with chloroform. The dry residue was weighed to calculate the yield. The residue was analysed to determine % potency of hydrocodone free base in it using assay methodology. For the cold water extraction test a suitable number of tablets were weighed and crushed in a coffee grinder. Cold water was added to the crushed powder and stirred well. The solution was kept in a freezer for suitable time and filtered slowly through a Whatman filter. The filtrate was collected and the active ingredient was analysed using assay methodology. For the syringeability and injectability test, the immediate-release abuse-resistant solid dosage form in the form of tablets of the present invention and the reference were weighed and crushed in a coffee grinder. A small amount of the powder was taken and heated in water on a spoon. The liquid from the spoon as drawn into an insulin syringe for a definite period of time. The difficulty of drawing the liquid into the syringe and the difficulty of expelling the solution from the syringe was recorded. The volume of the liquid drawn into the syringe known as syringe able volume and the volume of liquid expelled from the syringe known as injectable volume was measured. From the volume of contents of hydrocodone in the liquid expelled the amount recovered was calculated. For the simulated nasal fluid extraction test, a suitable number of tablets were weighed and crushed in a coffee grinder. A suitable quantity of the crushed powder was added to the simulated nasal fluid and shaken. Aliquots were withdrawn at various time points and the content of active pharmaceutical ingredient was analysed using assay methodology.

In one specific embodiment, the present invention provides an immediate release multiple pill abuse resistant solid dosage form that comprises
an intragranular phase containing melt extrudates of a mixture of hydrocodone or its pharmaceutically acceptable salt, release inhibiting agent which is a mixture of one or more reverse enteric polymer and one or more alkalizer, and one or more stabilizer and
an extragranular phase containing analgesic active ingredient and pharmaceutically acceptable excipient,
   wherein when more than the prescribed number of units of the immediate release multiple pill abuse resistant solid dosage form are orally administered, it provides at least reduced Cₘₐₓ as compared to the Cₘₐₓ achieved by administration of the conventional immediate release dosage form of hydrocodone or its pharmaceutically acceptable salt.

In one specific embodiment, the intragranular phase is in the form of extrudates prepared by hot melt extrusion. The intragranular phase contains hydrocodone or its pharmaceutically acceptable salt, release inhibiting agent composed of reverse enteric agent and an alkalizer and one or more stabilisers. The reverse enteric polymer of the release inhibiting agent is a copolymer based on dimethylaminoethyl methacrylate, butyl methacrylate, and methyl methacrylate or, co-polymer comprising methyl methacrylate (MMA) and diethylaminoethyl methacrylate. In this embodiment, a part of the alkalizer is present intragranularly and part of the alkalizer is present extragranularly. In very specific embodiment, the alkalizer is a combination of two different alkalizers for example, a mixture of magnesium oxide and sodium bicarbonate or carbonate.

In one specific embodiment, the present invention provides an immediate-release, abuse-resistant solid dosage form comprising
an intragranular phase containing melt extrudates of a mixture of hydrocodone or its pharmaceutically acceptable salt, one or more reverse enteric polymer, one or more alkalizer and one or more stabilizer and
an extragranular phase containing acetaminophen, alkalizer and pharmaceutically acceptable excipient,
   wherein when more than the prescribed number of units of the immediate release multiple pill abuse resistant solid dosage form are orally administered, it provides at least reduced Cₘₐₓ as compared to the Cₘₐₓ achieved by administration of conventional immediate release dosage form of hydrocodone or its pharmaceutically acceptable salt. In one specific embodiment, the intragranular phase is in the form of extrudates prepared by hot melt extrusion. The intragranular phase contains hydrocodone or its pharmaceutically acceptable salt, release inhibiting agent composed of reverse enteric agent and an alkalizer and one or more stabilisers. The reverse enteric polymer of the release inhibiting agent is a copolymer based on dimethylaminoethyl methacrylate, butyl methacrylate, and methyl methacrylate or, co-polymer comprising methyl methacrylate (MMA) and diethylaminoethyl methacrylate. In this embodiment, a part of the alkalizer is present intragranularly and part of the alkalizer is present extragranularly. In very specific embodiment, the alkalizer is a combination of two different alkalizers for example, a mixture of magnesium oxide and sodium bicarbonate or carbonate.

While the present invention is disclosed generally above, additional aspects are further discussed and illustrated with reference to the examples below. However, the examples are presented merely to illustrate the invention and should not be considered as limitations thereto.

### COMPARATIVE EXAMPLES

TABLE A
composition of the hot melt extrudates without any stabilizer

| | | |
|---|---|---|
| Composition of the Hot Melt | Example 1 | Example 2 |

| | |
|---|---|
| extrudates | (quantity in mg) |

| | | |
|---|---|---|
| Hydrocodone Bitartrate, USP | 10.0 | 10.0 |
| Methyl Methacrylate and | 120.0 | 120.0 |
| Diethylaminoethyl | | |
| Methacrylate Copolymer | | |
| Magnesium oxide | — | 7.0 |
| Sodium carbonate | — | 13.0 |
| Total weight | 130 | 150 |

Specified amounts of hydrocodone bitartrate, methyl methacrylate and diethylaminoethyl methacrylate copolymer (Kollicoat Smartseal 100 P), magnesium oxide USP (Light) and sodium carbonate NF (Anhydrous) were sifted through suitable sieve. The sifted ingredients were blended using suitable blender. The blend was subjected to melt extrusion at a temperature raising from about 90° C.-150° C. The extrudates were analysed for known and single highest individual unspecified impurity of hydrocodone.

The detection and quantification of the known and unspecified impurities of hydrocodone was performed by HPLC using a gradient programmed flow rate of 1 mL/minute with octadecyl silica column. The total known impurities such as hydrocodone aldol dimer and hydrocodone N-oxide and unspecified impurities were recorded at various relative retention times and quantified by external standard method.

The comparative Example 1 showed very high level total unspecified impurities of 1.884% and the single highest individual unspecified impurity was 0.643%. It was found that the comparative Example 2 showed very high level of total unspecified impurities were as high as 0.462% and the single highest unspecified impurity was 0.212%. This data indicated that the melt extrudates did not show acceptable impurity profile and there was a need of further stabilization.

### Examples 1 to 6 B

TABLE 1 (A)
Composition details of the hot melt extrudates of
the solid dosage form of the present invention

| Composition of the Hot Melt | Examples (weight in mg) | | | | | | | |
|---|---|---|---|---|---|---|---|---|
| extrudates | 1 | 2 | 3 | 4 | 5 | 6 | 6A | 6B |
| Hydrocodone Bitartrate, USP | 10.0 | | | | | | | |
| Methyl Methacrylate and | 120.0 | | | | | | 100.0 | |
| Diethylaminoethyl Methacrylate | | | | | | | | |
| Copolymer | | | | | | | | |
| Polyvinyl alcohol + polysorbate | 6.0 | 9.0 | 12.0 | 15.0 | 18.0 | 25 | — | — |
| 80 + polyethylene glycol (**) | | | | | | | | |
| Polyvinyl alcohol plus | — | — | — | — | — | — | 12.5 | |
| polyethylene glycol (#) | | | | | | | | |
| Polyvinyl alcohol -Partially | — | — | — | — | — | — | — | 12.5 |
| hydrolyzed | | | | | | | | |
| Magnesium Oxide USP (Light) | 5.0 | | | | | | | |
| Sodium Carbonate NF | 13.0 | | | | | | 10.8 | 10.8 |
| (Anhydrous) | | | | | | | | |
| Tartaric acid | — | | | | | | 3.0 | 3.0 |
| Butylated hydroxyl Anisole | — | | | | | | 0.17 | 0.16 |
| Weight of Hot melt Extrudes | 154 | 157 | 160 | 163 | 166 | 173 | 141.5 | 141.5 |

(#) Opadry ® II Clear 88F590009; PVA part hydrolyzed: 78% w/w, Macrogol PEG: m.w 3350 (22% w/w)
(**) Opadry ® II Clear 88F590007* (PVA): PVA part hydrolyzed: 75.66% w/w, Macrogol PEG: m.w 3350; 21.34% w/w; Polysorbate 80: 3% w/w

Specified amounts of hydrocodone bitartrate, methyl methacrylate and diethylaminoethyl methacrylate Copolymer, magnesium Oxide USP (Light) and sodium carbonate NF (Anhydrous) and polyvinyl alcohol, as mentioned in Table Number 1 (A), were sifted through suitable sieve. The sifted ingredients were blended using suitable blender. The blend was subjected to melt extrusion at a temperature raising from about 90° C.-150° C. The extrudates were analysed for the known and unspecified impurities of hydrocodone.

Examples of melt extrudates of 6A (polyethylene goycol) as well as 6B (devoid of polyethylene glycol) when subjected to stability studies, immediately upon extrusion, it was found that both examples, showed very low levels of known and unspecified impurities, the single highest individual unspecified impurity was 0.068% and 0.082%, respectively.

These melt extrudates in the form of intragranular phase were converted into solid dosage form as given below:

TABLE 1(B)
solid dosage form with intragranular phase of hot melt extrudates of hydrocodone
and extragranular phase of alkalizer and second active ingredient, acetaminophen.

| | Example numbers | | | | | | | |
|---|---|---|---|---|---|---|---|---|
| | 1A | 2A | 3A | 4A | 5A | 6A | 6AA | 6BA |
| | Quantity in mg | | | | | | | |
| Hot melt Extrudes of Table 1 A | 154 | 157 | 160 | 163 | 166 | 173 | 141.5 | 141.5 |
| Acetaminophen granules DC 90% | 361.1 | | | | | | | |
| Sodium bicarbonate | 85.0 | | | | | | | |
| Sodium Carbonate (Anhydrous) | 25.0 | | | | | | | |
| Polyethylene oxide mol wt. 30000 | 50.0 | | | | | | — | — |
| Magnesium Oxide USP (Light) | 90.0 | | | | | | 50 | 50.0 |
| Sodium Starch Glycolate Type A | 70 | 70 | 30.0 | 30.0 | 30 | 70 | — | — |
| Silicified Microcrystalline cellulose | 91 | 97.8 | 102.8 | 99.8 | 96.8 | 92 | 138.4 | 143.4 |
| Ferric oxide, NF (yellow) | — | — | — | 2. | 2 | 2 | — | — |
| Crospovidone, NF | 80 | | 60 | | | 80 | 60 | 60 |
| Colloidal silicon dioxide, NF | 10.0 | | | | | | | |
| Talc, USP | 7.0 | | | | | | | |
| Magnesium stearate, NF | 7.0 | | | | | | | |
| ***Film coating | — | | | | | | 35.4 | 35.6 |
| Total weight of the solid dosage form | 1030 | 1040 | 900 | 900 | 900 | 1050 | 920.4 | 925.6 |

* Polyox WSR N750: Polyethylene oxide mol wt 300000
***Film coating: Opadry Polyvinyl alcohol part hydrolysed, titanium dioxide, Macrogol/PEG3350. Talc.
@@ Quantitative composition of Acetaminophen granules DC 90%

TABLE 1C
Composition details of the acetaminophen granules

| Sr. No. | Ingredients | mg/Tab |
|---|---|---|
| 1 | Acetaminophen, USP | 325.0125 |
| 2 | Pregelatinized Starch, NF | 25.6400 |
| 3 | Povidone, USP, PVP K-30 | 5.0557 |
| 4 | Crospovidone, NF | 3.6110 |
| 5 | Stearic Acid, NF | 1.8056 |

Examples 1A to 6BA were prepared as follows: Hydrocodone bitartrate representing a drug susceptible to abuse, methyl methacrylate and diethylaminoethyl methacrylate copolymer (Kollioat Smartscal 100 P), mixture of polyvinyl alcohol polyethylene glycol and polysorbate 80, magnesium oxide USP (Light) and Sodium carbonate NF (Anhydrous) were sifted through suitable sieve. The above sifted ingredients were blended using suitable blender. In example 6AA and 6BA in addition to all the other excipients butylated hydroxyanisole and tartaric acid was added to the ingredients and the blend was hot melted extruded. Sifted ingredients were then processed using a hot melt extruder at suitable processing temperature and other parameters, hydrocodone bitartrate extrudates were collected. The melt extrudates of hydrocodone bitartarate were milled using suitable mill and passed through suitable sieve.

Intragranular phase of hydrocodone bitartrate extrudates were mixed with the extragranular phase. The extragranular phase was made up of acetaminophen granules, silicified microcrystalline cellulose (Prosolv SMCC 90), magnesium oxide, sodium bicarbonate, sodium carbonate (anhydrous), crospovidone and colloidal silicon dioxide, which was sifted separately through suitable sieves. Ferric oxide and talc were sifted together through suitable sieve. Magnesium stearate sifted separately through suitable sieve.

Solid Dosage Form Preparation:

Sifted acetaminophen granules, hydrocodone bitartrate extrudates and silicified microcrystalline cellulose were blended in a suitable blender. Subsequently, previously sifted ingredients, magnesium oxide, sodium bicarbonate, crospovidone, colloidal silicon dioxide, ferric oxide and talc were added to above blend in the blender and further blended. Finally, previously sifted magnesium stearate was added to above blend followed by lubrication blending. Compression of Hydrocodone bitartrate extrudates and acetaminophen dry mix blend was done. Lubricated blend was compressed. The compressed cores were film coated and were packed in High density polyethylene (HDPE) container with suitable desiccant.

The tablets prepared according to Example 6AA, 6BA, were subjected to accelerated stability studies and the impurities, both known and unspecified or unknown were quantified by High performance liquid chromatography.

Example 6AA which contained in the melt extrudates, apart from the stabilizers, polyethylene glycol. It was found that when converted into the solid dosage form with acetaminophen as second active ingredient, it was found that the highest individual unspecified single impurity was 0.22% which is beyond the acceptable limit. Thus, inspite of the presence of stabilizers which are polyvinyl alcohol, butylated hydroxyl anisole and tartaric acid, the presence of polyethylene glycol caused destabilization. This was concluded from the results of the stability of Example 6 BA which is devoid of polyethylene glycol. Example 6 BA was chemically robust and did not show any increase in the highest unspecified impurity limit of 0.2% by weight of the hydrocodone when quantified by high performance liquid chromatography. The total impurities also remained under control. The known impurities for eg. N-oxide impurity and the aldol dimer impurity also remained well within the acceptable limits led by International Council for Harmonisation (ICH), guidelines which are followed by the regulatory authorities like, EMEA, USFDA. The ICH provides guidance for the industry for various aspects, for eg. Stability Testing for New Dosage Forms.

The significance of presence of tartaric acid and butylated hydroxyl anisole in the melt extrudates was proved by the results of the solid dosage form having the melt extrudates prepared with polyvinyl alcohol, polyethylene glycol but devoid of tartaric acid and butylated hydroxyl anisole. This example provided highest unspecified impurity of less than 0.2% by weight of the hydrocodone base (0.057) after extrusion, but when formulated into a tablet dosage form with the extragranular phase having an alkalizer and acetaminophen, the tablets showed undesirable impurity level of N-Oxide of 0.546, at the end of three months and the highest impurities rose to as high as 0.459 at the end of three months when stored at 40° C./75% relative humidity, in sealed container. Whereas when the example 6B was formulated into a tablet dosage form 6BA butylated hydroxyanisole and tartaric acid in the melt extrudates, and an extragranular phase having an alkalizer and acetaminophen, the tablets showed an acceptable impurity level of known impurity level of N-Oxide of 0.058 at the end of three month, when stored at 40° C./75% relative humidity and the highest individual unspecified impurity was 0.162 at the end of three months, when stored at 40° C./75% relative humidity in sealed containers.

### Example 7

Solid dosage forms prepared according to Examples 1-6 and comparative example 1 were subjected to in vitro dissolution studies which are carried out in 0.01 N HCl 500 ml, in a Type II apparatus peak vessel at 50 rpm. Dissolution of multiple i.e. 4 tablets per vessel in 0.01N HCl+720 mg Citric acid 500 ml, using a Type II peak vessel at, 50 RPM, was also carried out The data compares the dissolution of 1 tablet in a 0.01 N HCl media vs. that of 4 tablets in 0.01 N HCl media and 0.01N HCl containing 720 mg Citric acid media for abuse proof capacity of the dosage form.

TABLE 2
Dissolution data of hydrocodone bitartrate tablet
in 0.01N HCl and 0.01N HCl and citric acid

| Time in | Examples | | | | | | |
|---|---|---|---|---|---|---|---|
| Min. | Comparative Ex-1 | Ex-1A | Ex-2A | Ex-3A | Ex-4A | Ex-5A | Ex-6AA |
| | % Dissolution of 1 tablet per vessel in 0.01N HCl | | | | | | |
| 5 | 35 | 38 | 37 | 40 | 53 | 43 | 60 |
| 10 | 50 | 55 | 53 | 57 | 70 | 62 | 75 |
| 15 | 58 | 65 | 64 | 68 | 79 | 73 | 83 |
| 30 | 71 | 80 | 78 | 84 | 91 | 89 | 92 |
| 45 | 78 | 88 | 87 | 91 | 95 | 95 | 96 |
| 60 | 83 | 92 | 90 | 95 | 97 | 97 | 98 |
| | % Dissolution of 4 tablets per vessel in 0.01N HCl | | | | | | |
| 5 | 8 | 13 | 12 | 15 | 20 | 18 | 27 |
| 10 | 12 | 19 | 17 | 21 | 26 | 25 | 34 |
| 15 | 14 | 23 | 21 | 26 | 30 | 29 | 39 |
| 30 | 18 | 29 | 27 | 33 | 37 | 36 | 46 |
| 45 | 21 | 32 | 31 | 37 | 40 | 40 | 50 |
| 60 | 22 | 35 | 33 | 39 | 42 | 43 | 53 |
| | % Dissolution of 4 tablets per vessel in 0.01N HCl + 720 mg Citric acid 500 ml | | | | | | |
| 5 | 14 | 21 | 13 | 22 | 22 | 18 | 39 |
| 10 | 19 | 28 | 18 | 28 | 30 | 26 | 47 |
| 15 | 22 | 33 | 24 | 35 | 38 | 34 | 52 |
| 30 | 28 | 42 | 35 | 48 | 50 | 48 | 61 |
| 45 | 32 | 48 | 42 | 52 | 56 | 54 | 66 |
| 60 | 35 | 52 | 46 | 55 | 61 | 59 | 70 |

The above dissolution data indicates that when more than the prescribed number of units of the immediate release multiple pill abuse resistant solid dosage form are orally administered, it provides a reduced Cₘₐₓ.

### Example 8A

### Effect of Coffee Grinding and Extraction Test

Solid dosage form prepared according to example 6BA and reference were subjected to extraction studies to evaluate ease of extracting hydrocodone bitartrate (HCB) from the intact and ground solid dosage for using water, at room temperature, water at 8° C. water at 85° C., 0.9% saline at 85° C. The reference used was the hydrocodone bitartarate acetaminophen tablets commercially available as Norco™. The procedure followed was as follows: Step a) A suitable number of tablets (5) were weighed and crushed in a coffee grinder for suitable time. Step b) The entire contents were transferred into a suitable flask and different extraction solvents listed above were added to the flask. Step c) The flask containing the ground tablets and solvent was shaken on a shaker for suitable time at suitable temperature. Step d) Aliquots were withdrawn at suitable time points and the content of hydrodocone analysed. The results obtained in various solvents were as follows:

TABLE 3
Percentage hydrocodone bitartarate extracted
in water at room temperature

| | Hydrocodone % extracted | | | | | | | |
|---|---|---|---|---|---|---|---|---|
| Extraction | water at room | | cold water | | water at | | Saline 0.9% | |
| medium | temperature | | at 8° C. | | 85° C. | | at 85° C. | |
| Time in | Ex | | Ex | | Ex | | Ex | |
| minutes | 6BA | Ref | 6BA | Ref | 6BA | Ref | 6BA | Ref |
| 5 | 6 | 59 | 2 | 58 | 30 | 77 | 27 | 95 |
| 10 | 12 | 64 | 4 | 63 | 46 | 81 | 37 | 95 |
| 15 | 15 | 67 | 4 | 63 | 57 | 82 | 43 | 94 |
| 30 | 21 | 70 | 6 | 66 | 66 | 86 | 52 | 94 |
| 60 | 27 | 77 | 9 | 70 | 74 | 88 | 63 | 96 |
| 120 | 32 | 83 | 12 | 74 | 76 | 88 | 73 | 96 |

The results indicate that upon grinding and subjecting the ground material to extraction in water at room temperature, example 6 BA released only 32% hydrocodone bitartarate as compared to 83% released by the reference product NORCO™. This indicates that the solid dosage form of the present invention provided a higher resistance to extraction. Upon grinding and subjecting the ground material to extraction in water at 8° C. example 6 BA released only 12% hydrocodone bitartarate as compared to 74% released by the reference product NORCO™. Also, it was found that solid dosage form provided resistance to extraction of hydrocodone bitartarate in water at 85° C. than that of the reference product. The same trend was exhibited when extracted with 0.9% saline at 85° C., in that only 73% hydrocodone bitartarate was extracted as compared to almost complete extraction of about 96% by the reference product.

### Example 8B

### Extraction of Free Base of Hydrocodone

The purpose of study was to extract the free base (Hydrocodone Bitratrate) from the formulation by pH adjustment followed by organic solvent extraction. Hydrocodone as free base is soluble in acetone, ethanol, ethyl acetate, and chloroform. Hydrocodone bitartrate (HCB) can be converted to free base by adjusting the pH of the solution towards alkaline pH 8-9. Thereafter, it can be extracted using organic solvent. The procedure followed for free base extraction was as follows:

Step a) Suitable number of tablets of the solid dosage form prepared as per the present invention and the reference Norco™ were weighed and crushed in a coffee grinder and powder transferred to a suitable beaker. Three samples of the example of the present invention and Norco™ were tested.
Step b) Warm water was added into beaker containing powder and stirred well.
Step c) The beaker was kept in a freezer for suitable time.
Step d) The solution was slowly filtered and filtrate collected and pH adjusted to 8-9 with suitable solution. Hydrocodone precipitated out in the solution as free base.
Step e) The dispersion was transferred into a separating funnel and extracted with chloroform and chloroform was evaporated.
Step f) The dry residue was weighed to calculate the yield.
Step g) The residue was analysed to determine % potency of hydrocodone free base in it using assay methodology. The tabulated data for percentage of free base extracted is as follows:

TABLE 4
percentage free base extraction data

| | |
|---|---|
| Example 6BA | Reference |

| | |
|---|---|
| Sample | % Hydrocodone bitartarate free base |

| | | |
|---|---|---|
| Average | 5.1 | 61.8 |

The percentage of hydrocodone bitartrate free base extracted from the solid dosage form prepared as per the example of the present invention was less only 5.1% as against 61.8% from the reference Norco™ which indicates the resistance of the solid dosage form to extraction in the free base form.

### Example 8C

### Cold Water Extraction

Cold water extraction has been reported as method which is widely used by abusers on opioid-acetaminophen combination products to separate acetaminophen from the product in order to increase the amount of hydrocodone that can be ingested to get a “high” while reducing at the same time the amount of acetaminophen which can potentially harm liver. Acetaminophen is reported as very slightly soluble in cold water and soluble in boiling water. While, Hydrocodone bitartrate (HCB) is reported as soluble in water. This solubility difference in cold water is utilized to separate acetaminophen from Hydrocodone bitartrate (HCB).

Procedure: Suitable number of tablets were weighed and crushed in a coffee grinder and powder transferred to a container. Cold refrigerated water was added to the crushed powder and solution was kept in freezer for suitable time and solution was then filtered. The active ingredient in the filtrate was analysed using assay methodology

TABLE 5A
Percentage of hydrocodone extracted in cold Water

| | |
|---|---|
| Example 6BA | Reference |

Hydrocodone % in the filtrate

| | |
|---|---|
| 22.4 | 64.2 |

The percentage of hydrocodone bitartrate (HCB) extracted from formulation of the present invention was less only 22.4% as compared to 64.2% extracted from the reference Norco™ in cold water extraction.

TABLE 5 B
Percentage of Acetaminophen extracted in cold Water

| | |
|---|---|
| Example 6BA | Reference |

Acetaminophen % in the filtrate

| | |
|---|---|
| 61.7 | 33 |

The percentage of acetaminophen extracted from solid dosage form of the present invention was around 60% as compared to 33 2% extracted from the reference Norco™ in cold water extraction. Higher amount of acetaminophen in the extract, may also deter the abuser from extracting and injecting with the aim of abuse, as acetaminophen may interfere and may therefore, present a deterrence.

### Example 9

### Syringeability and Injectability

Syringeability refers to ability of an injectable to easily pass through a hypodermic needle on transfer from a vial/container while Injectability refers to the ease of the injection during injection. Syringeability includes factors such as ease of withdrawal, clogging and foaming tendencies, and accuracy of dose measurements. Injectability includes pressure or force required for injection, evenness of flow, and freedom from clogging/blockage of the syringe needle.
a) Suitable number of tablets were weighed and crushed in a coffee grinder.
b) A small amount of crushed powder was added to water contained on a tablespoon and heat was applied for suitable time with intermittent stirring to obtain a suspension.
c) The suspension was drawn into an insulin syringe for a definite period.
d) Difficulty of drawing the suspension into the syringe was recorded
e) The volume of the suspension drawn into the syringe known as syringeable volume was recorded.
f) The suspension in the syringe was cooled to room temperature.
g) The suspension was expelled by injecting it into a small measuring cylinder and the volume expelled known as injectable volume was measured. The difficulty of expelling the suspension from the syringe and the difficulty of injecting was recorded.
h) The contents of hydrocodone in the suspension expelled were analysed and the amount recovered was calculated.

TABLE 6
Syringeability and Injectability data

| | Example 6BA | | | | Reference product .Norco ® | | |
|---|---|---|---|---|---|---|---|
| | Hydrocodone % | | | | Hydrocodone % | | |
| | Vol drawn | mg/vol | | | Volume drawn | mg/vol | |
| Sample | in 1 min | drawn | | Sample | in 1 min | drawn | |
| number | (ml) | (ml) | % | number | (ml) | (ml) | % |
| 1 | 0.7 | 0.38 | 26.9 | 1 | 1.6 | 2.16 | 67.4 |
| 2 | 1.2 | 0.50 | 20.6 | 2 | 2.1 | 3.06 | 73 |
| 3 | 1.4 | 0.65 | 23.3 | 3 | 1.8 | 2.75 | 76.4 |

| | |
|---|---|
| Observation: It was found that the it was moderately | Observation: It was found that the suspension was |
| difficult to draw the suspension into the syringe. | easy to inject. |

The percentage of hydrocodone bitartrate (mg/ml) which could expelled out of the syringe that is the injectable volume was less in formulation of the present invention as compared to the reference Norco™.

### Example 10

### Extraction in Simulated Nasal Fluid

The simulated nasal fluid extraction test was done to ascertain the ability of the solid dosage form to resist abuse through the nasal route by checking if the hydrocodone was released in the alkaline pH of the nasal fluid. The average baseline human nasal pH is approximately 6.3-6.4.

Suitable number of tablets were weighed and crushed in a coffee grinder The powdered mass was transferred to the simulated nasal fluid in a flask and shaken. Aliquots were withdrawn at various time points and analyze the content of hydrocodone using assay methodology.

TABLE 7
Simulated Nasal fluid extraction data

| | |
|---|---|
| Time in | % of hydrocodone extracted |

| minutes | Example 6BA | Reference NORCO ™ |
|---|---|---|
| 5 | 15 | 94 |
| 10 | 23 | 91 |
| 15 | 32 | 97 |
| 30 | 35 | 99 |
| 60 | 43 | 94 |
| 120 | 53 | 97 |

The results indicate that the solid dosage form of the present invention exhibited ability to resist extraction of hydrocodone bitartarate in the simulated nasal fluid. Only 53% of hydrocodone could be extracted in simulated nasal fluid as compared to 97% oh hydrocodone extracted from the the reference, or conventional solid dosage form.

1. An immediate-release, abuse-resistant solid dosage form comprising melt extrudates of a mixture of:
a) hydrocodone or its pharmaceutically acceptable salt,
b) release inhibiting agent comprising one or more reverse enteric polymer and one or more alkalizer and
c) one or more stabilizers.

2. An immediate-release, abuse-resistant solid dosage form of claim 1 wherein the extrudates form the first intragranular phase and the dosage form comprises a second intragranular phase comprising acetaminophen and pharmaceutically acceptable excipients and an extragranular phase comprising an alkalizer.

3. An immediate-release, abuse-resistant solid dosage form of claim 2, wherein hydrocodone salt is a bitartarate and each unit dosage form contains 5 mg to 20 mg of hydrocodone bitartarate.

4. An immediate-release, abuse-resistant solid dosage form of claim 1 wherein the stabilizer is vinyl containing polymer and is present in amount ranging from 1 to 20% by weight of the melt extrudates.

5. An immediate-release, abuse-resistant solid dosage form of claim 4 wherein the vinyl containing polymer is selected from polyvinyl acetate, polyvinyl alcohol, polyvinylcaprolactam-polyvinyl acetate-polyethylene glycol graft co-polymer, vinylpyrrolidone-vinyl acetate copolymers, copolymers of N-vinyl pyrrolidone and vinyl acetate or vinyl propionate, polyvinyl pyrrolidone, polyvinyl alcohol-polyethylene glycol-graft copolymers, copolymers of vinyl acetate and crotonic acid, partially hydrolyzed polyvinyl acetate, partially hydrolysed polyvinyl alcohol or mixtures thereof.

6. An immediate-release, abuse-resistant solid dosage form of claim 1 wherein the stabilizer is a mixture of vinyl containing polymer, butylated hydroxy anisole and organic acid.

7. An immediate-release, abuse-resistant, solid dosage form of claim 6 wherein the organic acid is selected from tartaric acid, citric acid, ascorbic acid or fumaric acid.

8. An immediate-release, abuse-resistant solid dosage form of claim 5 wherein the partially hydrolysed polyvinyl alcohol is present in amount ranging from 1 to 20% by weight of the melt extrudate, butylated hydroxyl anisole is present in amount ranging from 0.1 to 0.5% by weight of the melt extrudates and organic acid is present in amount ranging from 1 to 10% by weight of the melt extrudates, wherein hydrocodone bitartrate is present in amount ranging from 1 to 20% by weight of the melt extrudates.

9. An immediate-release, abuse-resistant solid dosage form comprising of claim 1 wherein the reverse enteric polymer is methyl methacrylate and diethylaminoethyl methacrylate copolymer.

10. An immediate-release, abuse-resistant solid dosage form of claim 3 wherein the weight ratio of hydrocodone bitartarate to polyvinyl alcohol is in the range 1:0.1 to 1:10.

11. An immediate-release, abuse-resistant solid dosage form of claim 3 wherein the weight ratio of hydrocodone bitartarate to reverse enteric polymer is in the range 1:1 to 1:20.

12. An immediate-release, abuse-resistant, solid dosage form of claim 1 wherein the melt extrudates contain one or more alkalizer selected from magnesium oxide, tromethamine, meglumine, calcium carbonate, sodium carbonate, sodium bicarbonate or mixtures thereof.

13. An immediate-release, abuse-resistant, solid dosage form of claim 1, wherein the dosage form comprises an extragranular phase comprising composition of second active ingredient selected from acetaminophen, naproxen, diclofenac or ibuprofen or their pharmaceutically acceptable salts.

14. A process of forming melt extrudates by mixing the hydrocodone or its pharmaceutically acceptable salt and reverse enteric polymer followed by heating the mixture to a temperature of from 60° C. to 220° C. to prepare an immediate release abuse resistant solid dosage form, a method of reducing degradation of hydrocodone resulting from the process, the method of reducing degradation of hydrocodone comprising performing the melt extrusion by subjecting the mixture of:
a) hydrocodone or its pharmaceutically acceptable salt,
b) release inhibiting agent comprising one or more reverse enteric polymer and one or more alkalizer and
c) one or more stabilizers;
followed by heating the mixture to a temperature of from 60° C. to 220° C.
